(19) United States
(12) Patent Application Publication
(10) Pub. No.: US 2005/0119682 A1
(43) Pub. Date: Jun. 2, 2005

(54) Vascular exclusion catheter

(76) Inventors: Eric Nguyen, Corona, CA (US); John R Brustad, Dana Point, CA (US); Matthew N Petrime, Los Angeles, CA (US); Gary M Johnson, Mission Viejo, CA (US); John A Cox, Macungie, PA (US); Charles C Hart, Summerville, SC (US)

Correspondence Address: APPLIED MEDICAL RESOUCES CORPORATION, 22872 Avenida Empresa, Rancho Santa Margarita, CA, 92688, US

(21) Appl. No.: 10/494,348

(22) PCT Filed: Sep. 10, 2002
(86) PCT No.: PCT/US02/28830

Related U.S. Application Data
(60) Provisional application No. 60/339,901, filed on Oct. 30, 2001.

(51) Int. Cl. A61M 29/00

(52) U.S. Cl. 606/194

(57) ABSTRACT

A fluid controlled device is adapted for disposition in a body conduit where it controls the flow of body fluids within the conduit. A sleeve is provided with a wall of separation having a first surface which defines a flow passage facilitating the flow of body fluids, and a second surface which defines an exclusion chamber sealed from the flow passage. Various dilators have properties for moving the seal between a low-profile state facilitating insertion of the device, any high-profile state defining the flow passage and exclusion chamber. The dilator may be skeletal, inflatable, and/or porous. An associated method includes the step of dilating a dilation assembly to move a wall from a low profile, insertible state, to a high-profile wherein the wall defines a flow passage and an exclusion cavity.

This is a non-provisional application claiming the benefits of Patent Convention Treaty Application Serial No. PCT/US02/28830 filed on Sep. 10, 2002 and entitled “Vascular Exclusion Catheter”; and U.S. Provisional Application Ser. No. 60/339,901 filed on Oct. 30, 2001 and entitled “Vascular Exclusion Catheter”, both of which are fully incorporated herein by reference.

### BACKGROUND OF THE INVENTION

1. Field of the Invention

This invention relates generally to apparatus and methods for at least partially occluding flow within a body conduit.

2. Discussion of the Relevant Art

Body conduits commonly provide for a flow of fluid from one location in the body to another location in the body. Typical of these fluid conduits are arteries and veins of the vascular system which provide a flow of blood between the heart and the organs of the body. When a particular procedure requires that the vessel be accessed, the flow of blood can be expected to exit the conduit through any access hole. This not only results in a loss of fluid such as blood, but also invades the general surgical environment with the fluid. In one such procedure, it is desirable to harvest the saphenous vein from the leg and to connect that vein to the ascending aorta in a Coronary Artery Bypass Grafting (CABG) procedure.

In the past, surgeons used an occlusion catheter to stop the flow of blood through the conduit or vessel. This catheter was provided with a spherical balloon which, when inflated, would totally obstruct the blood flow within the vessel. Particularly in the case of blood vessels, this is undesirable as an uninterrupted flow of blood is necessary to maintain the tissues of the body.

In order to avoid total occlusion, another procedure has been developed whereby the blood is totally removed upstream of the operative site and introduced down-stream of the operative site. In this procedure, commonly referred to as an “on-pump” (OPCABG) procedure, there is continuous uninterrupted beating of the heart. Nevertheless, this procedure requires management of blood flow from the aortotomy in order to create a viable proximal anastomosis. It is for this reason that many CAPD procedures are still performed off-pump.

Presently the surgeon's primary tool to accomplish sensation of blood flow from the aortotomy is a Partial Occluding Clamp. In these off-pump procedures, the Partial Occlusion Clamp is often used to engage the conduit or vessel exteriorly and thereby isolated a small portion of the vessel from the ongoing fluid flow.

While the partial occluding clamp is relatively simple to use, it is perceived by many to be very traumatic. Its use has been reported to cause secondary complications such as the fracturing of plague and resultant Transient Ischemic Attack or Cerebral Vascular Accident, with both local and global consequences. The partial occluding clamp also consumes much of the procedural area not only with its jaws on the aorta, but also with its clamp handles in the surgical field.

### SUMMARY OF THE INVENTION

These deficiencies of the past are overcome with the present invention which includes a catheter with a dilation assembly capable of maintaining fluid flow through a conduit while excluding a portion of the conduit from this fluid flow. Importantly, this catheter is non-invasive and is inserted endoluminally so that it does not require major space in the surgical environment. The dilation assembly of the catheter is capable of maintaining fluid flow within the conduit while producing an exclusion cavity that isolates a portion of the conduit from this fluid flow.

In one aspect the invention, a fluid-control device is adapted for disposition in a body conduit for controlling a flow of body fluids in the body conduit. The device includes a wall of separation having a first surface and an opposing second surface. The first surface defines a flow passage facilitating the flow of body fluids within the body conduit; and the second surface of the wall defines an exclusion chamber sealed from the flow passage and the flow of body fluids through the body conduit.

In another aspect of the invention, a catheter is adapted for disposition in a body conduit and includes a shaft which extends along an axis between a proximal end and a distal end. A dilation assembly is disposed at the distal end of the shaft and includes a first dilator operable to move between a high-profile state and a low-profile state. A second dilator is included in the assembly and is operable to move generally independently of the first dilator between the high-profile state and the low-profile state. A sleeve is carried by the first dilator and the second dilator between the high-profile state and the low-profile state.

In another aspect of the invention, a catheter is adapted for disposition in a body conduit. The catheter includes a shaft and a dilation assembly disposed at a distal end of the shaft. The dilation assembly has a low-profile state facilitating insertion of the assembly into the body conduit and a high-profile state facilitating operation of the assembly within the body conduit. The shaft includes an inner member which is disposed in a telescoping relationship with an outer member. A dilator has a first end carried by the outer member and a second end carried by the inner member. These first and second ends have a generally proximate relationship when the dilator is in the high-profile state and a generally spaced relationship when the dilator is in the low-profile state.

In another aspect of the invention, the dilation assembly includes a balloon that is inflatable to move the balloon to the high-profile state. In this state, first portions of the balloon define a fluid flow path to facilitate a flow of fluids in the body conduit.

In a further aspect, the invention includes an endovascular method for restricting blood flow along a predetermined area of a vessel without occluding blood flow through the vessel. This method includes the step of providing a catheter with a dilation assembly having a wall movable between a high-profile state and a low-profile state. The assembly is inserted into the vessel to an operative site in the low-profile state. At the operative site, the assembly is dilated to move the wall to the high-profile state where the wall defines with the predetermined area of the vessel an occlusion cavity isolated from the blood flow within the vessel.

These and other features and advantages of the invention will be further discussed with reference to preferred embodiments of the invention and reference to the associated drawings.

### DESCRIPTION OF THE DRAWINGS

FIG. 1 is an axial cross-section of a catheter having a dilation assembly in accordance with the present invention;

FIG. 2 is an axial cross-section view similar to FIG. 1 and illustrating the dilation assembly in a low-profile state;

FIG. 3 is an axial cross-section view showing the dilation assembly in a high-profile state and disposed within a body conduit;

FIG. 4 is an end view of the dilation assembly taken along lines 4-4 of FIG. 3;

FIG. 5 is an axial cross-section view similar to FIG. 3 and illustrating movement of the dilation assembly between the high-profile state and the low-profile state;

FIG. 6 is an axial cross-section view similar to FIG. 5 and illustrating a sleeve carried by dilators in a body conduit having a variable diameter;

FIG. 7 is an axial cross-section view similar to FIG. 6 and illustrating formation of a flow passage and in an exclusion cavity in accordance with the present invention;

FIG. 8 is an axial cross-section view of an additional embodiment wherein the exclusion cavity has an annular circumferential configuration;

FIG. 9 is a perspective view showing the embodiment of FIG. 8 in a body conduit;

FIG. 10 is an axial cross-section view showing use of the dilation assembly to occlude a secondary conduit without occluding a primary conduit;

FIG. 11 is a side-elevation view of a further embodiment of the invention;

FIG. 12 is a radial cross-section view taken along lines 12-12 of FIG. 11;

FIG. 13 is a perspective view of the embodiment of FIG. 11 showing an inflatable dilator in a high-profile state;

FIG. 14 is a perspective view of the dilator illustrated in FIG. 13 disposed in a body conduit;

FIG. 15 is a perspective view similar to FIG. 14 and showing dilation assembly of FIG. 11;

FIG. 16 illustrates placement of two sheets of material to form the inflatable dilator;

FIG. 17 illustrates the formation of heat seals to form seams of the balloon;

FIG. 18A illustrates a step for forming a seal line to define a lateral recess;

FIG. 18B is a radial cross-section view of the balloon taken along lines 18B-18B of FIG. 18A;

FIG. 19 is a perspective view of a further embodiment of the invention including circumferential connection lines;

FIG. 20 is a radial cross-section view taken along lines 20-20 of FIG. 19;

FIG. 21 is a further embodiment of the invention, including diagonal connection lines; and

FIG. 22 is a radial cross-section view taken along lines 22-22 of FIG. 21.

### DESCRIPTION OF PREFERRED EMBODIMENTS AND BEST MODE OF THE INVENTION

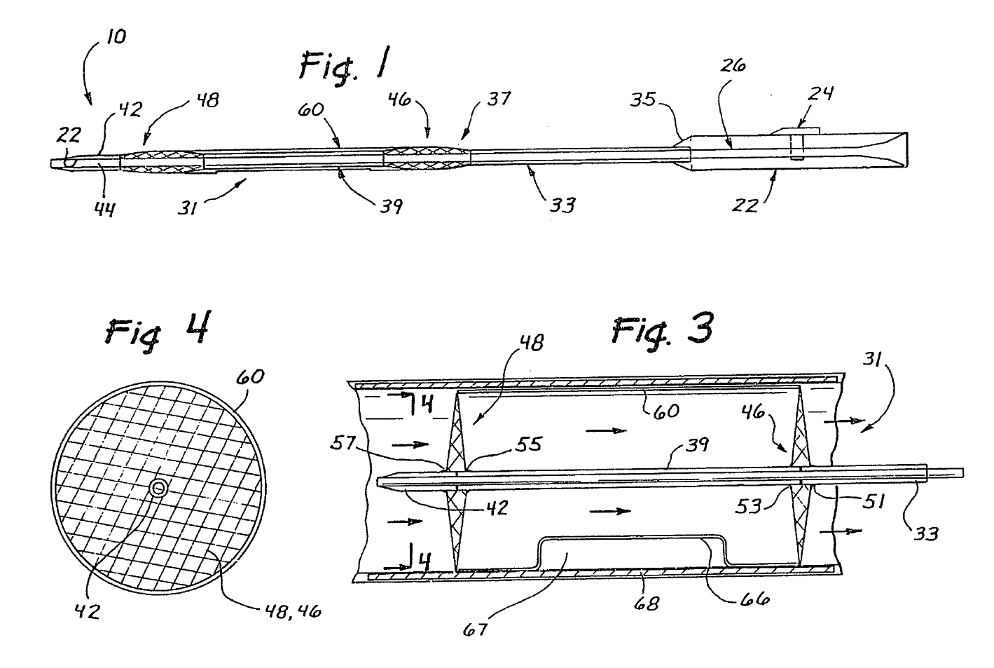

An exclusion catheter apparatus is illustrated in FIG. 1 and designated generally by the reference numeral 10. This particular apparatus 10 is adapted to exclude a segment of a body conduit while facilitating flow through the remainder of the conduit. The apparatus 10 comprises a handle assembly 20 with a hand piece 22 and an axially movable thumb slide 24. The thumb slide 24 is coupled to an inner elongate member 26 of a tube assembly 31. In the preferred embodiment, the inner elongate member 26 comprises a tube with a hollow core or lumen 27. Alternatively, the inner elongate member 26 may have a solid core and, thus, comprise a wire, for example.

The handle assembly 20 is coupled to the tube assembly 31, which in this embodiment comprises a first proximal outer tube 33 coupled to a distal portion 35 of the handle assembly 20. The inner elongate member 26 is disposed within the proximal outer tube 33, and extends distally outwardly from a distal tip 37 of the outer tube 33. A second floating outer tube 39 is disposed distally of the proximal outer tube 33 and is slidingly carried by the inner member 26. A third distal outer tube 42 is disposed distally of the floating outer tube 39 and secured to a distal portion 44 of the inner elongate member 26.

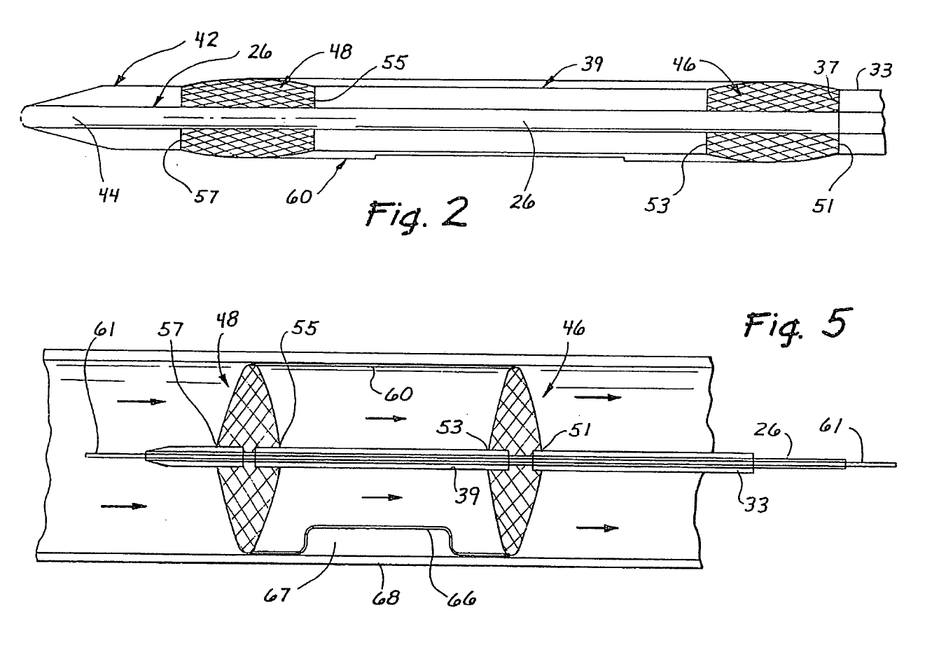

The tube assembly 31 includes a first proximal dilator 46 and a second distal dilator 48 which are movable between a low profile state, as illustrated in FIGS. 1 and 2, and a high profile state as illustrated in FIG. 3. The dilators 46, 48, are each provided with a permeable configuration in order to facilitate fluid flow through the dilators 46,48 in the high-profile state. In a preferred embodiment, each dilator 46, 48 comprises a braided tube which may be composed of a mesh of wires configured in a diamond or crisscross pattern as shown in FIG. 4. As best illustrated in the detail of FIG. 3, the proximal dilator 46 comprises a first dilator proximal end 51 secured to the proximal outer tube 33, and a first dilator distal end 53 secured to a proximal portion of the floating outer tube 39.

The distal dilator 48 comprises a second dilator proximal end 55 secured to a distal portion of the floating outer tube 39, and a second dilator distal end 57 secured to the distal outer tube 42. The ends of each dilator 46, 48 are configured to move with respect to each other in order to facilitate transition between a spaced-apart relationship, associated with the low profile state, and a proximate relationship, associated with the high profile state. It follows that the distance between the ends of each of the dilators 46, 48 determines the profile state of that dilator.

A sleeve 60 is coupled to the proximal dilator 46 and the distal dilator 48. The sleeve 60 surrounds the floating tube 39 and adjacent portions of the dilators 46, 48. In a preferred embodiment the sleeve 60 is composed of a thin-walled elastomeric material which is coupled to the dilators 46, 48 through a heat-sealing process. As fluid passes through the sleeve 60, the resulting fluid pressure expands the wall of the sleeve 60. An indented or recessed side portion 66 of the sleeve 60 is adapted to form an isolated exclusion chamber or recess 67 when the sleeve 60 is expanded to the high profile state. In an alternative embodiment, the sleeve 60 may omit the recess 67 and thus comprise an axially uniform cylinder.

In order to effect a low-profile state in the embodiment of FIGS. 1 and 2, the thumb slide 24 can be moved in a distal direction along the handpiece 22 causing the inner elongate member 26 to extend distally. Accordingly, the distal outer tube 42 is spaced apart from the floating outer tube 39 which is in turn spaced apart from the proximal outer tube 33. As these gaps are formed between the outer tubes 42, 39, 33, spaced-apart relationships are facilitated between first dilator proximal end 51 and the first dilator distal end 53, as well as between the second dilator proximal end 55 and the second dilator distal end 57.

The low-profile state of the dilators 46, 48 enables smoother introduction and removal of the apparatus 10 through body conduits, thereby minimizing trauma to the patient. Furthermore, the dilators 46, 48, and the sleeve 60 can be coated with an antithrombin agent and/or a hydrophilic coating to eliminate any potential thrombogenic response from the body conduit.

To effect a high-profile state of the dilators 46, 48, the thumb slide 24 is moved in a proximal direction along the handpiece 22, causing the inner elongate member 26 to move proximally. Fixed to the inner elongate member 26, the distal outer tube 42 also moves proximally carrying with it the second dilator distal end 57. The proximally directed force may also move the floating tube 39 in a proximal direction toward the proximal outer tube 33. As a result, the first dilator distal end 53 and the first dilator proximal end 51 move closer together. Similarly, the second dilator distal end 57 and the second dilator proximal end 55 also move closer together. Maximum dilation of the dilator 46, 48 may be achieved when the distal outer tube 42 is directed proximally to abut the floating tube 39, and when the floating tube is directed proximally to abut the proximal outer tube 33. In this configuration, the distal ends 53, 57 of the dilators 46, 48 are moved closely adjacent to the respective proximal ends 51, 55, as shown in FIG. 3. An incremental locking mechanism (not shown) may be provided on the thumb slide 24 to releasably lock each of the dilators 46, 48 to a preferred expanded diameter.

FIG. 5 illustrates two additional features which may be associated with the present invention. First, it will be noted that the elongate member 26 can be provided with the axial lumen 27 to facilitate insertion of the catheter apparatus 10 over a guidewire 61. Second, FIG. 5 illustrates that the catheter apparatus 10 can be used in a conduit which is smaller than the maximum diameter which can be achieved by the dilators 46 and 48.

In FIG. 5 it will be noted that these dilators, 46, 48 have expanded to meet the body conduit portion 68 and to carry the sleeve 60 into contact with this body conduit portion 68. This desirable result is achieved even though the dilators 46 and 48 have not been expanded to their maximum diameter as discussed with reference to in FIG. 4.

When the dilator 48 has a diameter less than its maximum diameter, it will also have an increased width along the axis of the catheter apparatus 10. This increased width is associated with a greater separation between the distal end of the floating outer tube 39, and the proximal end of the distal outer tube 42. Similarly, when the dilator 46 has a diameter less than its maximum diameter, it will have an increased width along the axis of the catheter apparatus 10 and greater separation between the distal end of the proximal outer tube 33 and the proximal end of the floating outer tube 39.

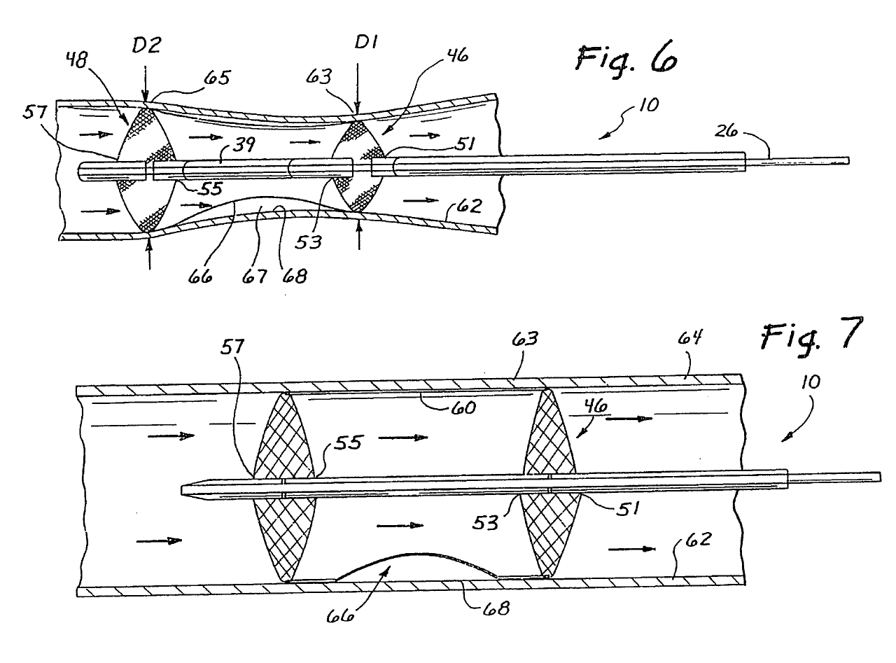

In FIGS. 6 and 7, the catheter apparatus 10 is illustrated in the body conduit 64. However in this case the conduit 64, more realistically, has a variable rather than a constant diameter. This is illustrated more specifically by the diameter D1 in proximity to the dilator 46, and the larger diameter D2 in proximity to the dilator 48. With the intent of maximizing flow through the sleeve 60, the catheter apparatus 10 is operable to move the sleeve 60 into contact with the inner wall 62 even when the conduit 64 has a variable diameter.

In operation, the elongate member 26 is moved proximally which initiates the process of expanding the dilators 46 and 48 as previously discussed. It is likely that only one of the dilators 46, 48 will expand until it contacts the inner wall 62. This will fix the floating outer tube 39 so that further proximal movement of the elongate member 26 will expand the diameter of the other dilator. In FIG. 6, the elongate member 26 is moved proximally along with the distal outer tube 42 and the floating outer tube 39. This closes the spacing between the proximal outer tube 33 and the floating outer tube 39, and accordingly increases the diameter of the dilator 46. When the dilator 46 reaches the diameter D1 of the inner wall 62, the movement of the floating outer tube 39 stops. The continued proximal movement of the elongate member 26 brings the distal outer tube 42 into closer proximity with the floating outer tube 39 thereby increasing the diameter of the dilator 48. The diameter of the dilator 48 will increase until it reaches the diameter D2 associated with the inner wall 62 at that location.

With reference to FIG. 7, it will be noted that the sleeve 60 is brought into contact with the inner wall 62, notwithstanding the variable diameters of the body conduit 64. Notably, this highly desirable feature is achieved because the dilators 46 and 48 can be provided with individual diameters that are independent of each other.

It will also be appreciated that full expansion of both dilators 46, 48 is accomplished when the force exerted against a first adjacent body wall by the first dilator 46 is equal to the force exerted against a second adjacent body wall by the second dilator 48. Therefore, in any body conduit wherein the diameters of the conduit portions adjacent to the dilators are not uniform, the self-adjusting characteristics of the apparatus 10 enable each dilator 46, 48 to expand to contact the respective adjacent portions with the same force.

As previously noted, the recessed sleeve portion 66 is radically spaced from the isolated body conduit portion 68 between the dilators 46, 48. The permeable dilators 46, 48 enable fluid to pass through the sleeve 60 with a resulting fluid pressure which distends the sleeve 60 to contact the inner wall 62 of the body conduit 64. Thus, the sleeve 60 facilitates flow through the body conduit 64 while isolating the particular body conduit portion 68. As a result, an isolated exclusion chamber 67 is defined by the recessed sleeve portion 66 and the isolated body conduit portion 68.

This optimizes the flow of fluid passing by the selected surgical site while the isolated portion 68 of the conduit remains excluded. Thus drugs, such as therapeutics, and fluids, such as irritants, may be delivered to or aspirated from the exposed conduit portion without risk of leakage into the isolated conduit portion 68. Tissue biopsy could also be obtained via the lateral access recess 66. An anastamosis or repair of the conduit portion 68 could also be performed while body fluid continues to flow through the remainder of the conduit 64. In particular, the body conduit portion 68 may be accessed exteriorly via a puncture, for example. Blood loss is minimized since only the volume contained in the isolated chamber 67 would be subject to loss. The sleeve 60 directs the passing fluid through the body conduit 62 and thus prevents any fluid communication between the flow channel of the sleeve 60 and the isolated chamber 67.

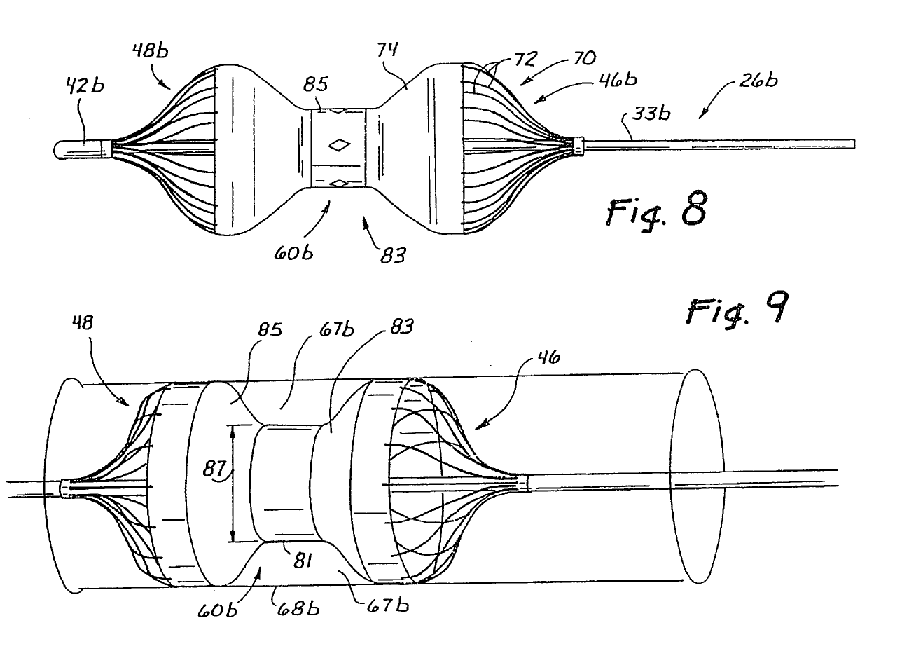

An additional embodiment of the invention is illustrated in FIGS. 8 and 9 where structural elements similar to those previously described are designated by the same reference numeral followed by the lower case letter “b.” Thus, in the embodiment of FIGS. 8 and 9, an alternative sleeve 60b is provided. The elongate member 26b in this embodiment includes the proximal outer tube 33b and the distal outer tube 42b which telescopes within the proximal outer tube 33b. A skeletal structure 70 is formed by a plurality of bendable members such as wires 72, each having two ends, one fixed to the outer proximal tube 33b and the other fixed to the distal outer tube 42b. With this construction, the distal outer tube 42b can be moved relative to the proximal outer tube 33b to provide the skeletal structure 70 with both a low-profile state and a high-profile state.

For example, if the distal outer tube 42b is moved distally of the proximal outer tube 33b, the ends of the wires 72 are widely separated. This causes the wires 72 to move into close proximity with the elongate member 26b in the low-profile state. However, when the distal outer tube 42b is moved proximally relative to the proximal outer tube 33b, the ends of the wires 72 become closely spaced. This causes the wire 72 to move generally radially to a high profile state as illustrated in FIGS. 8 and 9.

In order to form the sleeve 60b, a cover 74 is disposed over the skeletal structure 70. This cover 74 is typically formed of a distensible or elastomeric material and provided with a tubular configuration so that it at least partially covers the skeletal structure 70. At a central portion or waist 83, the cover 74 is provided with a collar or belt 85 which maintains the waist 83 at a reduced diameter in the high-profile state. As a result, the combination of the cover 74 and belt 85 provide the sleeve 60b with an hour-glass configuration. On either side of the belt 85, the cover 74 is free to expand to a relatively large diameter with the wires 72. However, at the central portion of the waist 83, the belt 85 limits this expansion to a reduced diameter.

Thus, the belt 85 provides the sleeve 60b with the recess 67b which in this case is formed circumferentially between the dilators 46b and 48b. When operatively disposed as illustrated in FIG. 9, the sleeve 60b isolates the body conduit portion 68b which in this case comprises a full 360 degree or circular portion of the body conduit 64b. Notwithstanding this isolated conduit portion 67b, the sleeve 60b is capable of continuing fluid flow within the body conduit 64b. Thus, a surgeon may exteriorly remove or puncture any part of the full circular conduit portion 68b without disrupting fluid flow through the remainder of the conduit 64b.

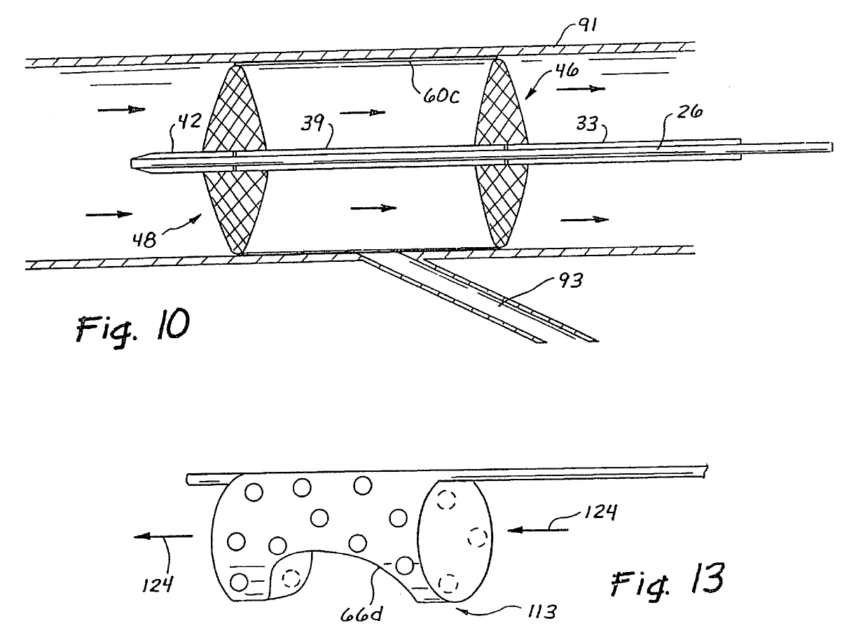

In some cases, it may not be necessary to operate on the isolated body conduit portion 68, but only to isolate the body conduit portion 68 from the flow in the main body conduit 64. In these instances, an embodiment such as that illustrated in FIG. 10 may be appropriate. In FIG. 10, elements of structure similar to those previously disposed are designated with the same reference numeral followed by the lower-case letter “c”. Thus, the sleeve 60c in this embodiment comprises an axial uniform cylinder which omits any recessed portion. In this case, the cylindrical sleeve 60c completely isolates a body conduit portion 91 which includes a branch conduit 93, for example. With the intent of merely isolating this branch conduit 93 from the flow in the main conduit 94c, there is no need for a recess such as that designated by the reference numeral 67b in the embodiment of FIG. 9.

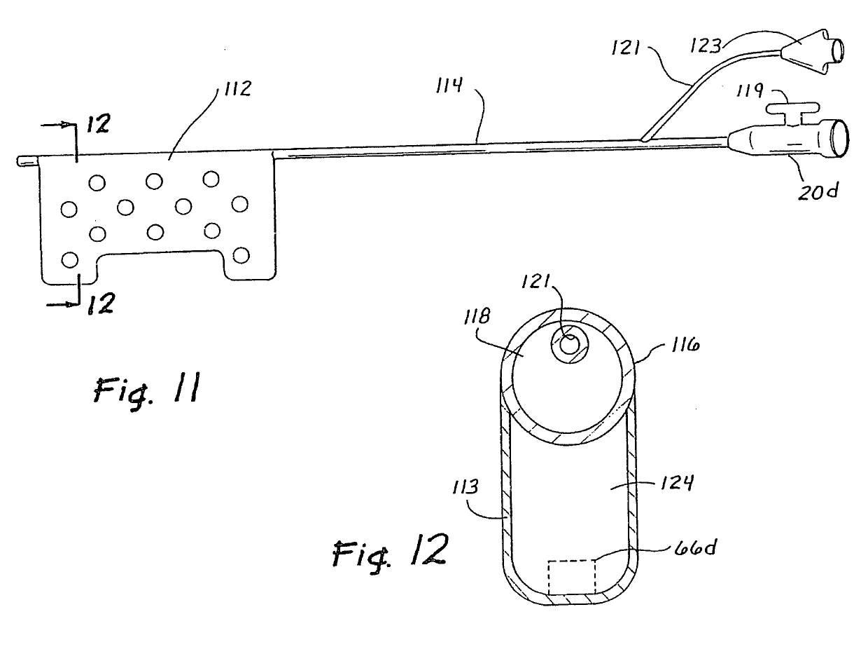

From the foregoing description, it will be apparent that the dilators 46 and 48 may comprise a variety of structures. In the embodiment of FIGS. 11-15, elements of structure similar to those previously discussed or designated with the same reference numeral followed by the lower-case letter “d.”

In FIGS. 11-13, the vascular exclusion catheter apparatus 10d includes a single inflatable dilator or balloon 112, that also serves as a dilating sleeve 113. This dilating sleeve 113 is coupled to a catheter shaft 114 that extends from the handle assembly 20d. The shaft 114 comprises an outer tube 116 which in this case defines a relatively large through-lumen 118. The through-lumen 118 is sized and configured to receive a standard guidewire which can be used to place the catheter apparatus 10d and to otherwise orient the dilating sleeve 112 at the operative site.

The handle assembly 20d includes a stopcock 119 which controls access to the through-lumen 118. An inner tube 121 having an inflation lumen 122 accessible through an inflation port 123, is coupled to a proximal portion of the outer tube 116. In a preferred embodiment, the inner tube 121 extends only partially along the through-lumen 118 terminating within the through-lumen 118 in proximity to the dilating sleeve 112. Thus, an inflation gas exiting from the inflation lumen 122 is directed through the through-lumen 118 into the dilating sleeve 112.

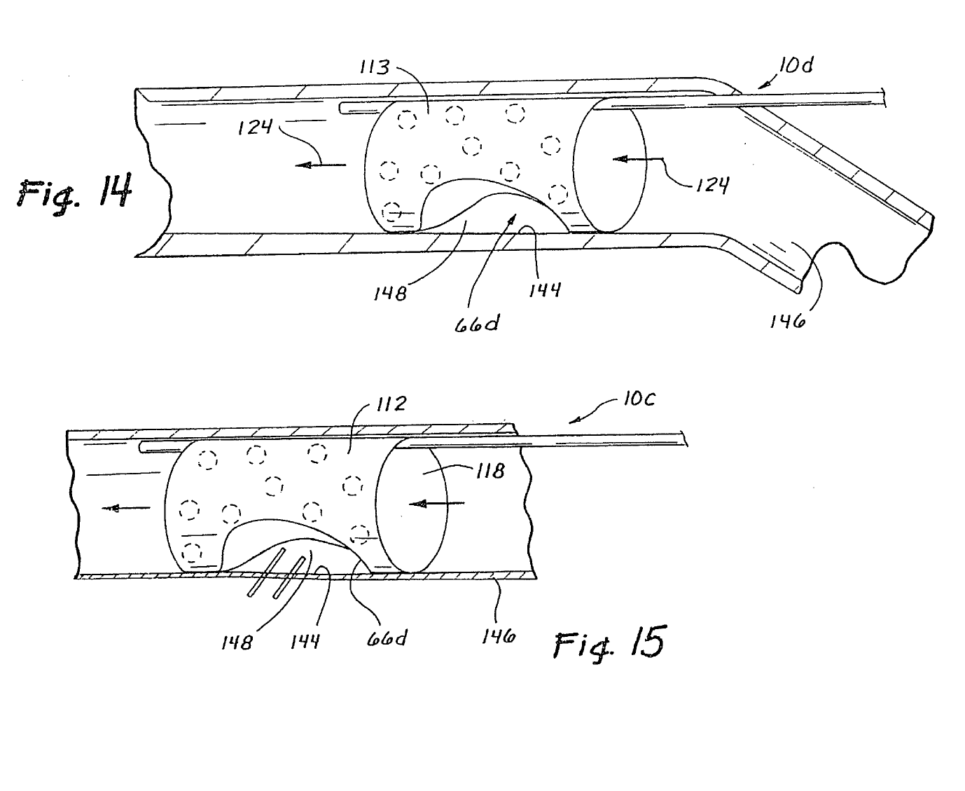

In this manner, gas from the inflation lumen 123 inflates the balloon or dilating sleeve 112 to a high profile state. In this state, the sleeve is circumferentially inflated but defines an axial flow passage shown by the arrows 124 in FIG. 13.

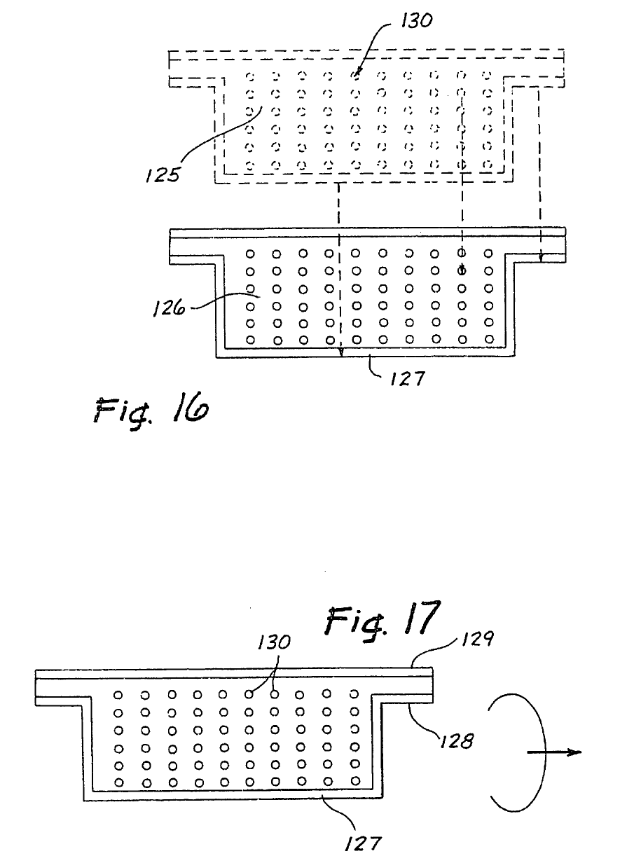

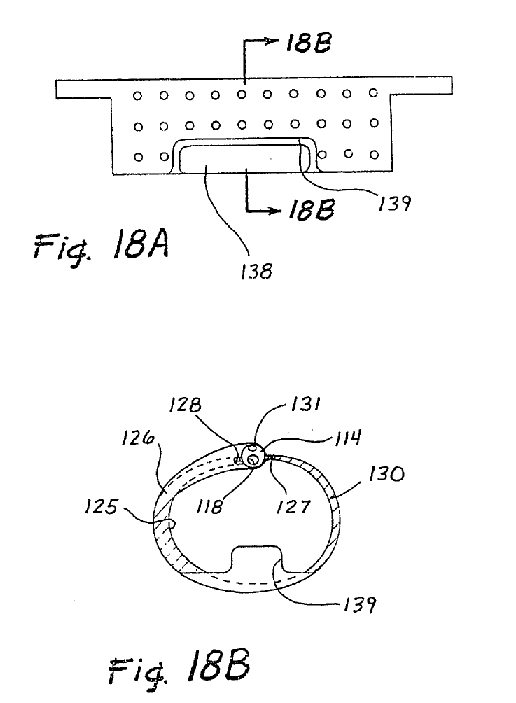

A preferred method of constructing the balloon 112 is illustrated in FIGS. 16, 17, 18a and 18b. In accordance with this method, the balloon 112 is formed of two layers, 125 and 126, of thermoplastic material, each sealed or otherwise joined together, for example, along seams 127, 128 and 129. The layers 125 and 126 can also be spot welded at a plurality of layer-joining connection points 130. With this construction, the balloon 112 is formed between the layers 125 and 126 and bounded by the seams 127-129. The catheter shaft 114 can be inserted between, and sealed to the seams 127 and 128. This gives the catheter shaft 114, and particularly the inflation lumen 122 access to the interior of the balloon 112 between the layers 125 and 126. With this construction, the balloon 112 can be formed into the cylindrical configuration of the sleeve 113 by rolling the layers 125 and 126 back on themselves and attaching the seam 127 to the seam 129 as illustrated in FIG. 18b. In this embodiment, if the recess 66d is desired, it can be formed by removing a portion 138 as illustrated in FIG. 18a and forming a seal of 139 to join the four edges of the layers 125 and 126.

As illustrated in FIG. 18b, the catheter shaft 114 can be formed with multiple lumens, namely, the through-lumen 118 and the inflation lumen 122. With this construction, at least one skive 131 can be cut in the shaft 114 to access the inflation lumen 122. In operation, the inflation gas will pass from the inflation lumen 122 through the skive 131 and into the balloon 112 between the layers 125 and 126.

With further reference to FIGS. 14 and 15, it will be noted that the dilating sleeve 113 facilitates maximum fluid flow while excluding or isolating a specific area 144 of a body conduit 146 to form an isolated chamber 148. The lateral recess 66d facilitates access to a portion of the body conduit for fluid or therapeutic administration, tissue biopsy, anastomosis procedure, or for repairing damage while body fluid continues to flow through the conduit. As with previous embodiments, the apparatus 10d may be introduced to the surgical site through either percutaneous or direct access.

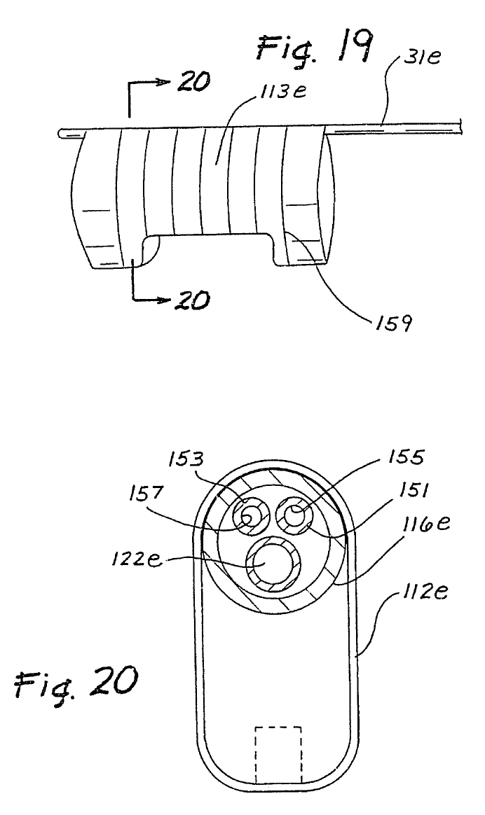

In an alternate embodiment shown in FIGS. 19 and 20, elements of structure similar to those previously disclosed are designated with the same reference numeral followed by the lower-case letter “e.” Thus, this embodiment includes the tube assembly 31e, the balloon 112e, the outer tube 116e, and the inflation lumen 122e. However, in this case additional tubes 151, 153 are disposed within the tube 116. These additional tubes 151, 153 provide further lumens 155, 157, respectively, for fluid administration. Also, the dilating sleeve 113e comprises an inner balloon layer joined to the outer balloon layer via transverse connection lines 159, instead of the connection points 130 shown in the embodiment of FIGS. 16-18.

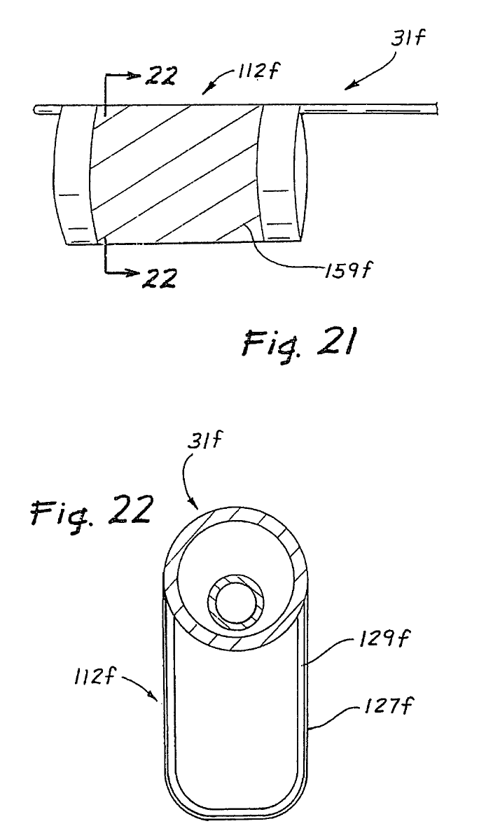

In a further embodiment, illustrated in FIGS. 21 and 22, elements of structure similar to those previously disclosed are designated with the same reference numeral followed by the lower-case letter “f.” Thus, this embodiment includes the tube assembly 31f, the balloon 112f and the connection lines 159f. In this case, however, the dilating sleeve 112f may be formed without any recess 67 (FIG. 5) and thus may comprise an axially uniformed cylinder.

Furthermore, the outer layer 126f of the balloon 112f may be provided with a lesser thickness than the inner layer 125f thereof. This difference in layer thickness facilitates expansion of the balloon 112f toward the thinner area upon inflation. Thus, as the outer layer 127f is expanded, the inner layer 129f is uniformly pulled along with the outer layer.

With the specific disclosure of the foregoing embodiments, it will be apparent that many alterations and modifications can be made without departing from the spirit and scope of the invention. It is for this reason that the illustrated embodiments are set forth only as examples and should not be taken as limiting the invention.

The words used in this specification to describe the invention and its various embodiments are to be understood not only in the sense of their commonly defined meanings, but to include by special definition in this specification the generic structure, material or acts of which they represent a single species.

1. A fluid control device adapted for disposition in a body conduit for controlling a flow of body fluids in the body conduit, the device comprising:
a wall of separation having a first surface and a second surface opposing the first surface;
the first surface of the wall defines a flow passage facilitating the flow of body fluids within the body conduit; and
the second surface of the wall defines an exclusion chamber sealed from the flow passage and the flow of fluids through the body conduit.

2. The fluid control device recited in claim 1, further comprising a sleeve having properties for defining the flow passage with the first surface of the wall.

3. The fluid control device recited in claim 2 further comprising at least one dilator having properties for moving the sleeve between a low-profile state facilitating insertion of the device into the body conduit, and a high-profile state facilitating the flow of fluids through the body conduit.

4. The fluid control device recited in claim 1, wherein the exclusion chamber is defined by the second surface of the wall exteriorly of the sleeve.

5. The fluid control device recited in claim 3, wherein the dilator is inflatable.

6. The fluid control device recited in claim 3, wherein the dilator comprises a skeleton.

7. The fluid control device recited in claim 6, wherein the skeleton is formed of wires.

8. The fluid control device recited in claim 6, wherein the exclusion chamber has an annular configuration.

9. The fluid control device recited in claim 8, wherein the dilator has a waist and the wall includes a belt disposed around the waist of the dilator.

10. The fluid control device recited in claim 8, wherein the exclusion chamber is disposed circumferentially of the dilator.

11. A catheter adapted for disposition in a body conduit, comprising:
a shaft having an elongate configuration and extending along an axis between a proximal end and a distal end;
a dilation assembly disposed at the distal end of the shaft;
a first dilator included in the dilation assembly and being operable to move between a high-profile state and a low-profile state;
a second dilator included in the dilation assembly and being operable to move generally independently of the first dilator between the high-profile state and the low-profile state; and
a sleeve carried by the first dilator and the second dilator between the high-profile state and the low-profile state.

12. The catheter recited in claim 11, wherein portions of the sleeve define an exclusion cavity.

13. The catheter recited in claim 11, wherein the first and second dilators are generally porus to facilitate a flow of fluid within the body conduit and through the dilation assembly.

14. The catheter recited in claim 12, wherein the first and second dilators are generally porus to facilitate a flow of fluid within the body conduit and through the dilation assembly exclusive of the exclusion cavity.

15. A catheter adapted for disposition in a body conduit, comprising:
a shaft having an elongate configuration and extending along an axis between a proximal end and a distal end;
a dilation assembly disposed at the distal end of the shaft and having a low-profile state facilitating insertion of the assembly into the body conduit and a high-profile state facilitating operation of the assembly within the body conduit;
an outer member included in the shaft;
an inner member included in the shaft and disposed in a telescoping relationship with the outer member;
a dilator included in the dilation assembly, the dilator having a first end carried by the outer member and a second end carried by the inner member;
the first end and the second end having a generally proximate relationship when the dilator is in the high-profile state; and
the first and second end having a generally spaced relation-ship when the dilator is in the low-profile state.

16. The catheter recited in claim 15, wherein the dilator is a first dilator and the catheter further comprises:
a proximal member included in the shaft, the proximal member disposed outwardly of the inner member and proximally of the outer member;
a second dilator having a first end carried by the outer member and a second end carried by the proximal member; whereby,
proximal movement of the inner member relative to the proximal member moves the first dilator to the high profile state and moves the second dilator to the high profile state independently of the first dilator.

17. The catheter recited in claim 16, wherein:
the outer member floats on the inner member of the shaft to facilitate the independent movement of the first dilator relative to the second dilator.

18. The catheter recited in claim 17, wherein the first dilator and the second dilator are generally porus to fluids flowing in the body conduit.

19. A catheter adapted for disposition in a body conduit, the catheter comprising:
a shaft having an elongate configuration and extending along an axis between a proximal end and a distal end;
a dilation assembly disposed generally at the distal end of the catheter shaft and having a low-profile state facilitating insertion of the assembly into the body conduit, and a high-profile state facilitating operation of the assembly within the body conduit;
a balloon included in the dilation assembly;
the balloon being inflatable to move the balloon to the high-profile state; and
first portions of balloon in the high-profile state defining a fluid flow path to facilitate a flow of fluids in the body conduit.

20. The catheter recited in claim 19, wherein the balloon has a generally cylindrical configuration and extends generally circumferentially of the fluid flow path.

21. The catheter recited in claim 20, further comprising:
second portions of the balloon in the high-profile state defining an exclusion cavity outside the fluid flow path to inhibit the flow of fluids through the exclusion cavity.

22. The catheter recited in claim 19, wherein the shaft includes a through-lumen extending through the balloon, and an inflation lumen terminating within the balloon.

23. The catheter recited in claim 20, wherein the balloon includes;
a first sheet;
a second sheet sealed to the first sheet to form an inflation cavity, and
the first sheet and the second sheet being bent back on themselves to provide the balloon with the cylindrical configuration.

24. An endovascular method for restricting blood flow along a predetermined area of a vessel without occluding blood flow through the vessel, comprising the steps of;
providing a catheter having a dilation assembly with a wall movable between a high-profile state and a low-profile state;
inserting the dilation assembly into the vessel to an operative site in the low-profile state;
dilating the dilation assembly to move the wall to the high-profile state, whereby:
the wall in the high-profile state defines with the predetermined area of the vessel an exclusion cavity isolated from blood flow within the vessel.

25. The endovascular method recited in claim 24, wherein the providing step includes the step of:
providing the wall with a first side and a second side;
placing the wall at a location within the dilation assembly wherein the first side of the wall defines the exclusion cavity and the second side of the wall defines a blood flow path sealed from the exclusion cavity.

26. The endovascular method recited in claim 25 wherein the providing step includes the step of:
forming the dilation assembly with a skeleton moveable between the high-profile state and the low-profile state; and
attaching a sleeve to the skeleton to move the sleeve between the high-profile state and the low-profile state.

27. The endovascular method recited in claim 25 wherein the dilating step includes the step of inflating the dilation assembly to move the wall to the high-profile state.

28. The endovascular method recited in claim 27, wherein:
the providing step includes the step of providing the dilation assembly with a balloon; and
the dilating step includes the step of inflating the balloon to move the wall to the high profile state.

29. A method of making an inflatable dilation assembly, comprising the steps of:
providing a first piece of sheet material;
sealing a second piece of sheet material to the first piece of sheet material along at least one perimeter seam to form an inflation cavity; and
bending the first piece of sheet material and the second piece of sheet material back on themselves to provide the inflation cavity with a generally cylindrical configuration.

30. The method recited in claim 29, wherein the sealing step includes the step of forming a first perimeter seam and a second perimeter seam spaced from the first perimeter scene.

31. The method recited in claim 30 further comprising the steps of:
attaching the first perimeter seam to the second perimeter seam to maintain the inflation cavity in the cylindrical configuration.

32. The method recited in claim 30, wherein the forming step includes the step of forming the first perimeter seam in a generally parallel relationship with the second perimeter seam.
